(19) United States
(12) Patent Application Publication
(10) Pub. No.: US 2014/0188252 A1
(43) Pub. Date: Jul. 3, 2014

(54) POLYCENTRIC KNEE JOINT PROSTHESIS FOR EXTREME AFFORDABILITY

(71) Applicants: Joel Sadler, San Francisco, CA (US); Vinesh Narayan, Oakland, CA (US); Eric Thorsell, Portland, OR (US)

(72) Inventors: Joel Sadler, San Francisco, CA (US); Vinesh Narayan, Oakland, CA (US); Eric Thorsell, Portland, OR (US)

(73) Assignee: D-Rev: Design for the Other Ninety Percent, San Francisco, CA (US)

(21) Appl. No.: 14/002,088

(22) PCT Filed: Mar. 5, 2012
(86) PCT No.: PCT/US2012/027766

§ 371 Date: Feb. 4, 2014

Related U.S. Application Data
(60) Provisional application No. 61/464,382, filed on Mar. 3, 2011.

(51) Int. Cl. A61F 2/64

(52) U.S. Cl. CPC A61F 2/644
USPC 623/46; 623/39

(57) ABSTRACT

An above knee prosthesis comprises an upper block, a lower block, a middle linkage pivotably coupling the middle posterior regions of the upper and lower blocks together, and at least one side linkage pivotably coupling the sides of the upper and lower blocks together. The center of rotation of the prosthesis is located above the prosthesis when it is in full extension and moves downward as the prosthesis rotates into full flexion. When the prosthesis is in use and in full extension, a majority of the weight of the patient borne by the prosthesis is directly transferred from the upper block to the lower block. A bumper disposed between the upper and lower blocks will typically be provided to absorb shock and dampen noise when the prosthesis is in extension. A leaf spring may be provided to bias the prosthesis to be in extension.

### CROSS-REFERENCE

This application claims the benefit of U.S. Provisional Application No. 61/464,382, filed Mar. 3, 2011, the entire contents of which are incorporated herein by reference.

### BACKGROUND OF THE INVENTION

Above knee prostheses are useful in providing mobility to amputees who would otherwise have difficulty with ambulation. Typically, these devices include an upper “socket” to connect to the user's leg, a “pylon” that forms the lower leg and interfaces with a foot prosthesis, and the knee joint itself. Because knee joints must endure substantial forces, they are typically constructed of very strong, durable materials and designed with precision to reduce wear. As a result, current knee prostheses are very expensive and generally not accessible to amputees in the developing world. Thus, there is a need in the prosthetics field for an above knee prosthesis that has been designed for extreme affordability.

Patents and patent publications which may be of interest include: U.S. Pat. Nos. 2,208,275, 3,820,169, 3,823,424, 4,005,496, 4,064,569, 4,145,766, 4,215,442, 4,310,932, 4,756,713, 4,911,709, 5,171,325, 5,800,567, 6,749,640, 6,752,835, 7,066,964, 7,087,090, 7,279,010, and 7,544,214; and U.S. Pub. No. 2010/0082115. Scientific publications which may be of interest include: Blumentritt, S., Scherer, H. W., Wellershaus, U., Michael, J. W., 1997, “Design principles, biomechanical data and clinical experience with a polycentric knee offering controlled stance phase knee flexion: a preliminary report,” Journal of Prosthetics and Orthotics 9:1, 18; Chakraborty, 1994, “A new modular six-bar linkage trans-femoral prosthesis for walking and squatting,” Prosthetics and Orthotics International 18:2; Gard, S. A., Childress, D. S., Uellendahl, J. E., 1996, “The Influence of the Four-Bar Linkage Knees on Prosthetic Swing-Phase Floor Clearance,” Journal of Prosthetics and Orthotics 8:2:34-40; Greene, M. P., 1983, “Four Bar Linkage Knee Analysis,” Prosthetics and Orthotics International 37:15-24; Paul, J. P., 1999, “Strength requirements for internal and external prostheses,” Journal of Biomechanics 32: 381-393; Radcliffe, C. W., 1994, “Four-bar linkage prosthetic knee mechanisms: kinematics, alignment and prescription criteria,” Prosthetics and Orthotics International 18:159-73.

### SUMMARY OF THE INVENTION

This invention relates generally to the prosthetics field, and more specifically to highly functional above knee prostheses designed for extreme affordability, for example, by being made from relatively simple yet durable components and designed for extended wear.

A first aspect of the invention provides an above knee prosthesis comprising an upper block, a lower block, a middle linkage, and at least one side linkage. The above knee prosthesis has a fully extended configuration and a fully flexed configuration. The upper block has a bottom face, and the lower block has a top face. In many embodiments, the upper block has a rounded front face and/or a domed top face, and the lower block may comprise a cylindrical main body and/or an internal clamp or socket for receiving a pylon that interfaces with a foot prosthesis. The middle linkage pivotably couples a middle posterior region of the upper block with a middle posterior region of the lower block. The side linkage(s) pivotably couples a side of the upper block with a side of the lower block. The above knee prosthesis is “polycentric,” e.g., the center of rotation of the lower block relative to the upper block is located above the prosthesis when the prosthesis is in the fully extended configuration and moves downward as the prosthesis rotates from the fully extended configuration to the fully flexed configuration. When the prosthesis is in use in a patient and in the fully extended configuration, a majority of the weight of the patient borne by the prosthesis is directly transferred from the bottom face of the upper block to the top face of the lower block. In many embodiments, the upper block, the lower block, the middle linkage, and the side linkage(s) are made of relatively soft and light-weight material such as a polymer, e.g., nylon 6-6.

The above knee prosthesis will typically include two side linkages, a first side linkage and a second side linkage. The first side linkage is disposed on a first side of the upper block and on a first side of the lower block. The second side linkage is disposed on a second side of the upper block opposite the first side of the upper block and on a second side of the lower block opposite the first side of the lower block. The prosthesis may further comprise a cap coupling the first side linkage with the second side linkage, the cap being disposed in front of an anterior portion of the upper block and an anterior portion of the lower block much like a knee-cap.

Typically, the middle linkage pivotably couples an internal middle posterior region of the upper block with an internal middle posterior region of the lower block. Also, the side linkages are typically disposed external of a side of the upper block and a side of the lower block. In other embodiments, the middle linkage may instead be external and/or the at least one side linkage may be internal.

The linkages will typically take the form of bars having first and second ends. The middle linkage may comprise a bar having a first end and a second end, the first end being pivotably coupled to the middle posterior region of the upper block and the second end being pivotably coupled to the middle posterior region of the lower block. The middle linkage may be pivotably coupled to the upper and lower blocks in many ways. For example, a pin that traverses through-holes in the upper block and the first end of the middle linkage and a pin that traverses through-holes in the lower block and the second end of the middle linkage may be provided. Each side linkage comprises a bar having a first end and a second end, the first end being pivotably coupled to the side of the upper block and the second end being pivotably coupled to the side of the lower block. The side linkage may be pivotably coupled to the upper and lower blocks in many ways. For example, a pin that traverses through-holes in the upper block and the first end of the side linkage and a pin that traverses through-holes in the lower block and the second end of the side linkage may be provided. In this manner, the above knee prosthesis can have a “four-bar linkage geometry.”

The bottom face of the upper block and the top face of the lower block have interfaces with each other such that a majority of the weight of the patient borne by the prosthesis is directly transferred from the bottom face of the upper block to the top face of the lower block. For example, the bottom face of the upper block and the top face of the lower block comprise flat surfaces, curved surfaces, pegs, roller bearings, or other interfaces that allow the majority of the weight of the patient borne by the prosthesis to be directly transferred from the bottom face of the upper block to the top face of the lower block. Typically, the bottom face of the upper block and the upper face of the lower block are flat and/or comprise low friction surfaces.

When the prosthesis is in the fully extended configuration, the upper block will typically be angled relative to the lower block at an angle of 0 degrees. When the prosthesis is in the fully flexed configuration, the upper block will typically be angled relative to the lower block at an angle of 165 degrees. The prosthesis may be configured to have other angles of full extension and/or flexion.

In many embodiments, the above knee prosthesis further comprises a bumper disposed between the bottom face of the upper block and the top face of the lower block. The bumper is adapted to absorb shock and dampen noise when the prosthesis is placed into the fully extended configuration. The bumper may be coupled to the bottom face of the upper block, preferably removeably coupled, for example, by a bolt which may be elongated such that it also couples a base member to the top face of the upper block. The bumper may be adjustable to adjust the distance between the bottom face of the upper block and the top face of the lower block. The bumper may be adjustable to adjust the angle of the upper block relative to the lower block when the prosthesis is placed into the fully extended configuration. The bumper may have an angled or stepped bottom face. The bumper may be made of a soft, compliant material, e.g., polyurethane or rubber, and have a durometer range of 70-90 (preferably 85) on the shore A hardness scale. In some embodiments, the bumper comprises two or more layers of materials with variable stiffness such that the harder, more resilient material contacts the lower block first and the subsequent layers are more compliant to absorb shock.

In many embodiments, the prosthesis is biased to be in the fully extended configuration. For example, the prosthesis may further comprise a leaf spring coupling the upper block to the lower block and adapted to bias the prosthesis to be in the fully extended configuration. The leaf spring may be disposed internally in the upper block and the lower block. The leaf spring may comprise a flat leaf spring, typically comprising spring steel. In some embodiments, the leaf spring is instead coupled to the back of the middle linkage.

The prosthesis may further comprise a gravity-activated latch adapted to ensure that the prosthesis remains in the fully extended configuration during the stance phase of a patient's gait. For example, such a latch could comprise a latch pivotably coupled to the upper block and which is pulled against a peg coupled to the lower block. In the stance phase, the latch and peg are coupled together to ensure that the prosthesis stays extended. When the user has raised the knee prosthesis to step forward, gravity can pull the latch away from the peg and release the knee prosthesis from extension.

Another aspect of the invention provides a system for replacing a leg of an amputee patient from above the knee. The system comprises the above knee prosthesis according to the first aspect of the invention, a rounded socket adapted to fit to a stump of the leg of the patient, an inner disc adapted to couple to the interior of the socket and to the above knee prosthesis to secure the prosthesis relative to the socket, and an outer disc adapted to couple to the exterior of the socket and to the above knee prosthesis to secure the prosthesis relative to the socket. A portion of socket is sandwiched between the inner and outer discs.

In many embodiments, the system further comprises n elongated bolt adjusted to adjustably couple the upper block of the above knee prosthesis, the outer disc, the rounded socket, and the inner disc together. The system may further comprise a bumper adjustably coupled to the bottom face of the upper block of the above knee prosthesis by the elongated bolt

### BRIEF DESCRIPTION OF THE FIGURES

FIG. 1A is a perspective view of an exemplary above knee prosthesis according to embodiments of the present invention.

FIG. 1B is a front view of the above knee prosthesis of FIG. 1A.

FIG. 1C is a side view of the above knee prosthesis of FIG. 1A.

FIG. 1D is another perspective view of the above knee prosthesis of FIG. 1A.

FIGS. 2A-2C are side views of joint rotation and the resulting change in center of rotation in the above knee prosthesis of FIG. 1A.

FIGS. 3A and 3B are side views of the upper and lower blocks of the above knee prosthesis of FIG. 1A.

FIG. 4 is a side view of a bumper between the upper and lower blocks in the above knee prosthesis of FIG. 1A.

FIGS. 5A-5C are side views of potential connection interfaces to the socket and pylon for the above knee prosthesis of FIG. 1A.

FIG. 6 is a side and perspective view of a split-clamp mechanism for the above knee prosthesis of FIG. 1A.

FIGS. 7A and 7B are perspective and side views, respectively of the side links combined to form a kneecap in an exemplary above knee prosthesis according to another embodiment of the present invention.

FIG. 8 is a graph to illustrate the effect of geometry on the dynamic center of rotation.

FIG. 9A is a transparent perspective view of the knee prosthesis of FIG. 1A to illustrate nuts and bolts and a modular pyramid interface.

FIG. 9B is an exploded, transparent view of the knee prosthesis of FIG. 1A to illustrate nuts and bolts and a modular pyramid interface.

FIG. 10 is a perspective view of a bumper assembly and an embodiment of the adjustable stability mechanism for above knee prostheses according to embodiments of the present invention.

FIGS. 11A-11C are side and internal views of a stepped wedge adjustable stability mechanism for above knee prostheses according to embodiments of the present invention.

FIGS. 12A and 12B are side and perspective views, respectively, of the leaf spring extension assist mechanism.

FIG. 13A is a cross-sectional, exploded view of an exemplary socket attachment disc system.

FIG. 13B is a cross-sectional view of the socket attachment disc system of FIG. 13A.

FIGS. 13C and 13D show various components of the socket attachment disc system of FIG. 13A.

FIG. 14 is a side view of an exemplary above knee prosthesis having a gravity-assisted latch according to embodiments of the present invention.

FIGS. 15A-15H show an above knee prosthesis according to another embodiment of the present invention.

### DESCRIPTION OF THE PREFERRED EMBODIMENTS

The following description of the preferred embodiments of the invention is not intended to limit the invention to these preferred embodiments, but rather to enable anyone skilled in the art of prosthetics to make and use this invention.

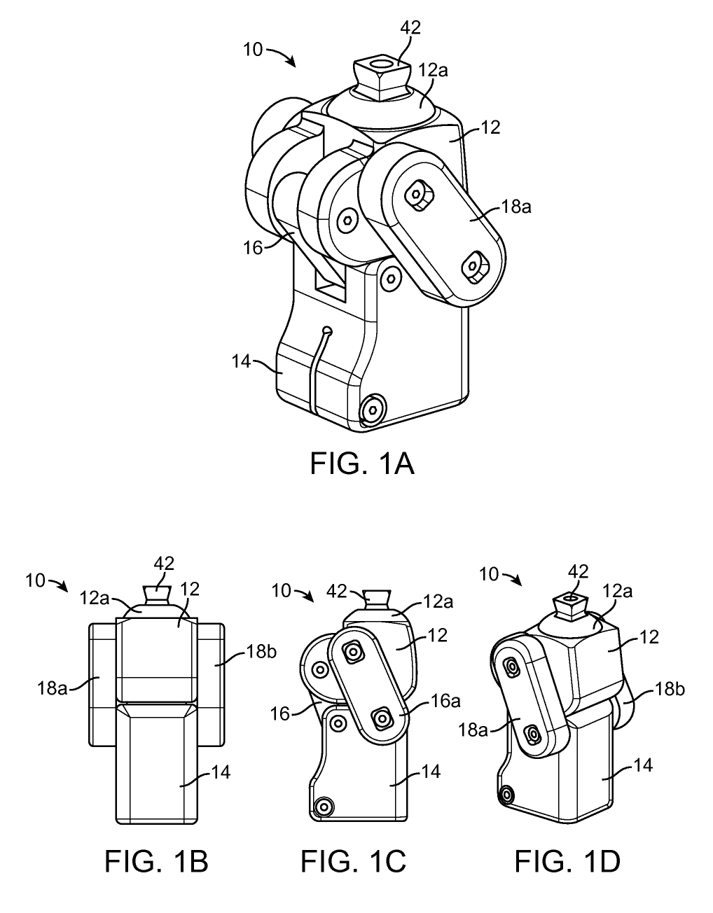

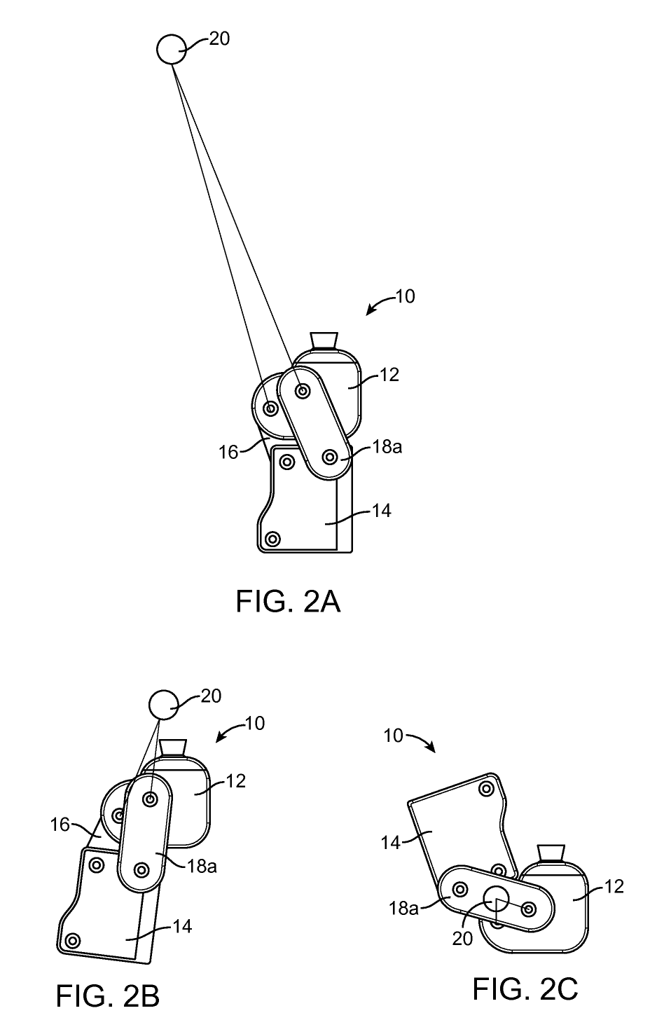

As shown in FIGS. 1A-1D, an above knee prosthesis 10 primarily comprises an upper block 12, a lower block 14, two side linkages 18a and 18b, and middle linkage 16. FIG. 1A shows a perspective view of the rear and right side of the prosthesis 10. FIG. 1B shows a front view of the prosthesis 10. FIG. 1C shows the right side of the prosthesis 10. FIG. 1D shows a perspective view of the front and right side of the prosthesis 10. The prosthesis 10 works as illustrated in FIGS. 2A-2C, where the user's natural gait provides rotational forces that move the prosthesis from full extension as shown in FIG. 2A to full flexion as shown in FIG. 2C. As a result of four-bar linkage geometry, the center of rotation 20 for the prosthesis 10 is located above the prosthesis 10 in the stance phase as shown by FIG. 2A, and moves dynamically downward as the prosthesis rotates as shown by FIGS. 2B and 2C. This “polycentric” design of the knee prosthesis results in a high level of stability without the need for complex mechanisms such as hydraulics or microcontrollers. The full range of motion for this joint is preferably 0-165 degrees, with 0 degrees being the angle of full extension as shown by FIG. 2A and 165 degrees being the angle of full flexion as shown by FIG. 2C.

Alternatively, the above knee prosthesis 10 may include more or less numbers of linkages, such as only a single side linkage and one mid linkage.

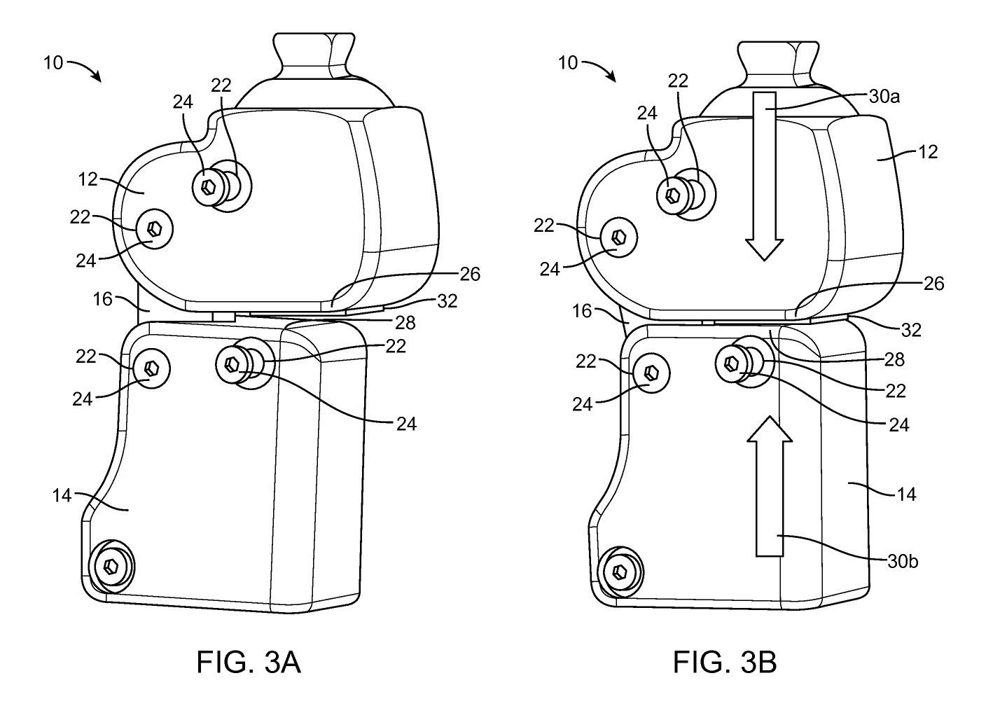

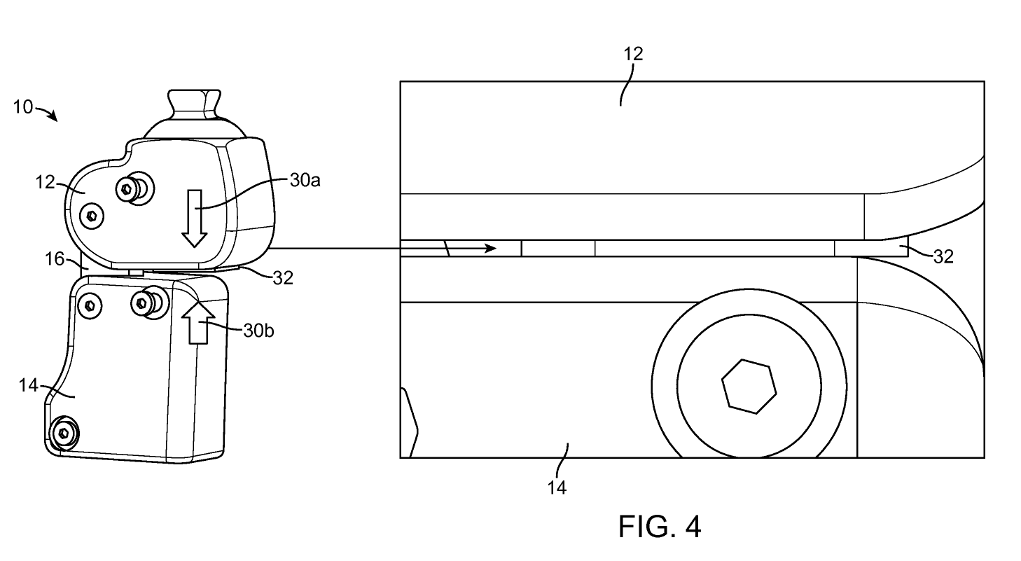

As shown in FIGS. 3A and 3B, the upper block 12 and the lower block 14 of the knee prosthesis 10 each have two through-holes 22 that accept connectors 24, such as bolts, which attach the blocks 12, 14 to the side linkages 18a, 18b and middle linkage 16. For the ease of viewing these through-holes 22 and connectors 24, the knee prosthesis 10 is shown without the side linkages 18a, 18b in FIGS. 3A to 3B. In the preferred embodiment, the blocks 12, 14 interface with the side linkages 18a, 18b externally and the middle linkage 16 internally, but the linkages 16, 18a, and 18b could be either internal or external to the blocks 12, 14. The method of load transfer from the upper block 12 to the lower block 14 during extension is direct, with the bottom face 26 of the upper block 12 resting on the top face 28 of the lower block 14 during stance phase, as shown in FIG. 3B. Use of direct load transfer from the upper block 12 to the lower block 14 significantly reduces the loads on the side linkages 18a, 18b and the middle linkage 16. Without direct load transfer, these linkages 16, 18a, and 18b would either have to be constructed of a very strong material or designed to be much larger than presented in the preferred embodiments. Thus, the use of direct load transfer can avoid these alternatives, resulting in a knee joint prosthesis that can be produced for extreme affordability. In place of flat faces 26 and 28 on the upper block 12 and lower block 14, respectively, that interface with one another to produce this result, the surfaces could also be curved, make use of pegs, include roller bearings, or involve any other kind of interface that would allow for direct load transfer. Direct load transfer is illustrated by the use of force arrows 30a and 30b as shown in FIGS. 3B and 4.

Furthermore, the direct interface can make use of a bumper 32 between the upper block 12 and the lower block 14 to absorb shock and dampen noise when the knee prosthesis 10 reaches full extension, as shown in FIG. 4. This part of the gait is called terminal impact, and without a bumper 32 in place, it can create an uncomfortable “knocking” experience for the user. The bumper 32 is preferably constructed of a soft, compliant material, such as a polyurethane or rubber. In addition to providing shock absorption and noise dampening, this bumper 32 also provides a small amount of compliance in the joint prosthesis 10 during stance phase, which reduces the forces that are transferred directly to the user's socket. This small amount of compliance is sometimes referred to as stance phase flexion, and is a desirable feature of prosthetic knee joints.

The bumper 32 may be comprised of a single piece of compliant material or multiple composite materials of different mechanical properties such as stiffness. It is desirable to have a material that is soft enough to absorb shock and dampen noise, but also mechanically resistant to wear. Typically a more compliant material will have reduced wear properties. In exemplary embodiments, the bumper 32 will have a durometer range of 70-90 (preferably 85) on the shore A hardness scale and/or be made of polyurethane or rubber. An alternative bumper 32 may be made of two or more layers of variable stiffness material such that the harder more resilient material contacts the lower block first and the subsequent layers are more compliant to absorb shock. This allows a composite bumper 32 that can include a relatively soft material as well as a protective hard layer.

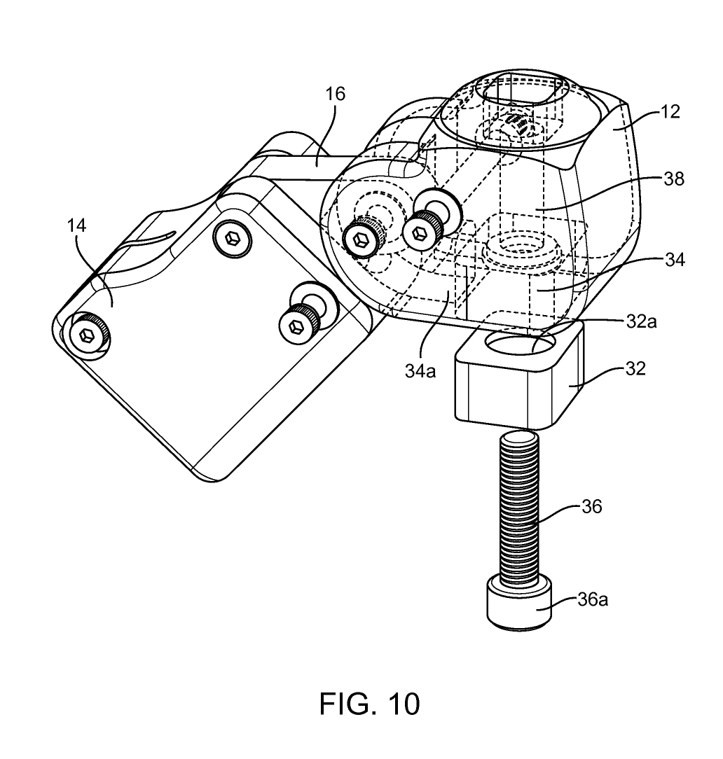

The bumper 32 may be mechanically fixed in the upper block 12 by means of an attachment mechanism to prevent movement. FIG. 10 shows a preferred attachment mechanism for a bumper 32 where the bumper 32 fits into keyed rectangular slot 34 in the upper block 12 and is fixed in place with a single bolt 36. A counter-sunk through-hole 32a in the bumper 32 allows the attachment bolt 36 to pass through and constrain the bumper 32 from movement. The head of the attachment bolt 36a may be nested within the bumper 32. The remainder of the attachment bolt 36 will be threaded into threaded channel 38. The attachment bolt 36 then may thread into a nut or the threaded modular pyramid component 40. An advantage of this bumper geometry is that the bolt 36 may be accessed easily externally so that removal and adjustments can be made to the bumper assembly without needing to remove the knee prosthesis 10 from the socket or pylon attachment interfaces to the patient's leg and foot prosthesis. Therefore, dynamic adjustment of the bumper 32 is possible at the time of fitting. The single bumper attachment bolt 36 may fix multiple parts in the upper joint assembly so that the number of parts is reduced. In its preferred embodiment the modular pyramid adapter 40 acts as a threaded nut to the bumper attachment bolt 36, which also fixes other parts on its axis, such as the bumper 32 as well as washers.

The integrated bumper 32, modular attachment mechanism, and attachment bolt 36 in the upper block 12 will typically provide better load transfer in the knee joint assembly by allowing more force to directly transfer through the bumper assembly rather than in the weaker linkages or upper joint material.

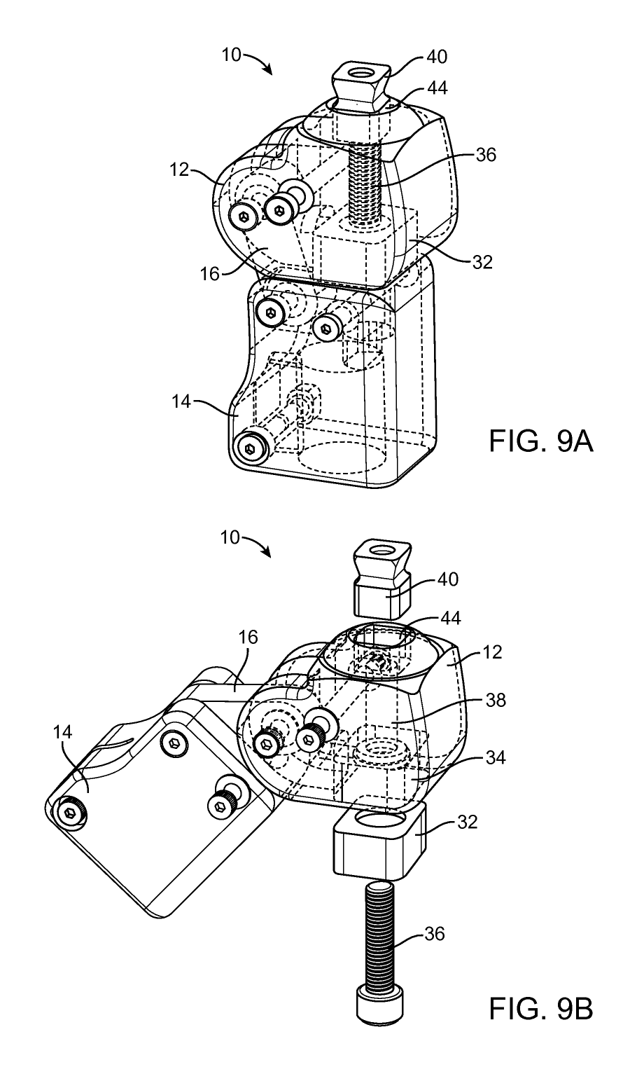

The upper block 12 and the lower block 14 also have connection mechanisms for attachment to the socket and lower limb pylon. The connection mechanism for the socket is shown in FIG. 1 as a pyramid adapter 42 that is integrated directly into the upper block 12. Use of such an integrated connection allows for modularity, whereby other types of adapters could also be used. A keyed hole in the upper block 12 may accept different types of modular adapters to allow attachment to the prosthetic socket. The preferred embodiment of the invention integrates a base-less pyramid 40 that is inserted inside a keyed slot 44 in the top of the upper block 12. FIGS. 9A and 9B show the pyramid component 40 and corresponding keyed slot 44 in the upper block 12. In this embodiment, the pyramid 40 may be held in place with a fastening feature such as a threaded bolt 36 in upper block 12 assembly.

Typically pyramid adapters used in modular prosthetics have a large integrated base to more evenly distribute applied loads. The domed portion 12a of the upper block 12 (as shown by FIGS. 1A to 1D), in which the pyramid 40 or 42 is inserted, provides a mechanical support to the pyramid 40 or 42 so that it may not need a large base. This base-less pyramid has the advantage of a reduced size and weight. Since corresponding pyramid components in many existing above knee prostheses are often made of hard materials such as steel, aluminum or titanium, any reduction in volume is desirable from size, weight and cost perspective.

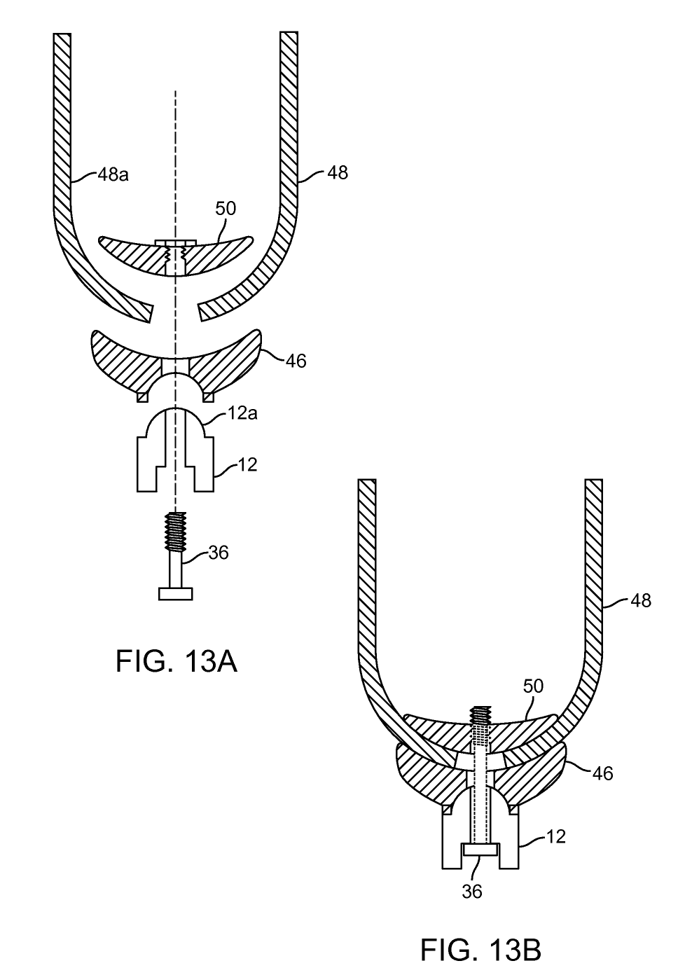

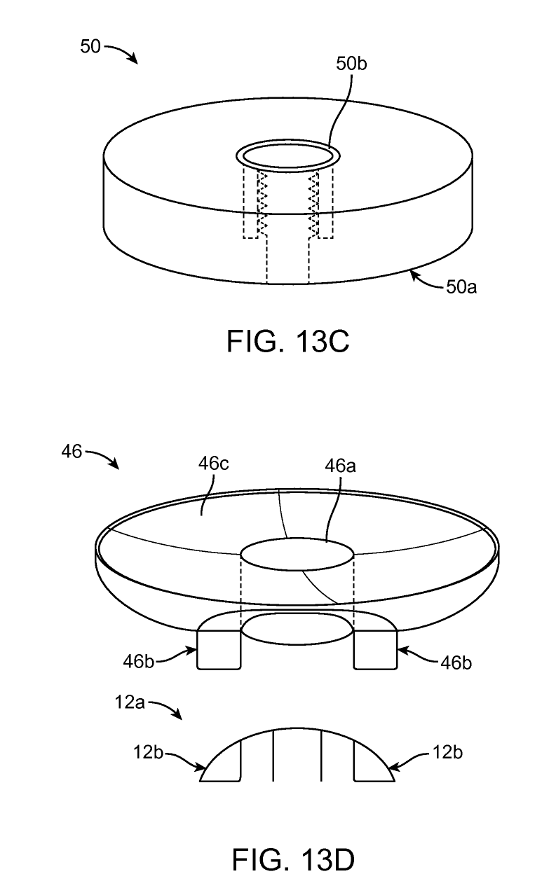

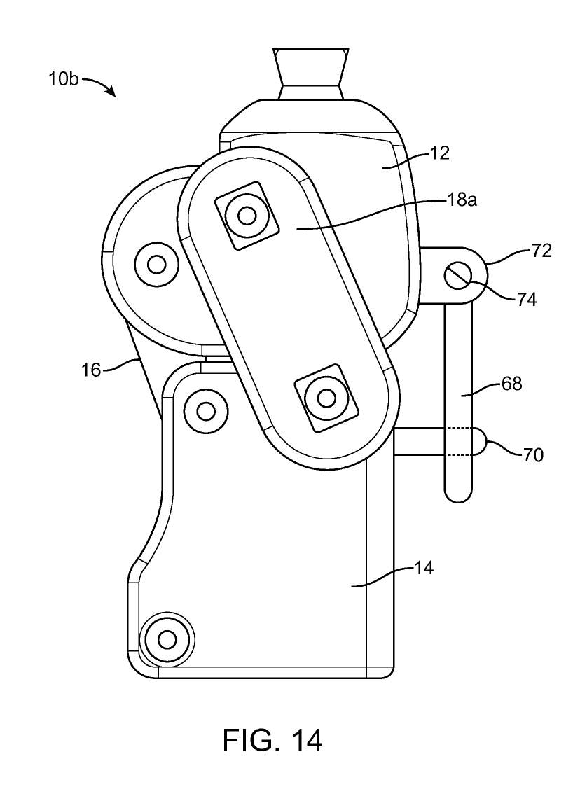

An alternative to a modular pyramid socket interface is a disc attachment component 46 shown in FIGS. 13A to 13D. The socket attachment disc 46 has a curved upper surface 46c so as to conform to the shape of the socket 48 as well as the domed shape top 12a of the upper block 12. This disc component 46 may be mechanically constrained to the upper block 12 by means of a keyed interface with an attachment bolt 36 passing through threaded or slotted hole 46a. Mating features 12b on the upper block 12 receive rotational locking features 46b from the outer socket disc 46. On the inside of the socket 48, an additional disc 50 has a lower surface 50a conforming to the inner wall 48a of the socket 48 so that the socket 48 is mechanically pressed between the outer socket attachment disc 46 and inner socket attachment disc 50. The entire assembly of the upper block 12, outer disc 46, socket 48, and inner disc 50 is held together in compression with an attachment bolt 36 that threads into a feature in the inner socket disc 50, such as an embedded nut 50b. Additional mechanical constraints may be added by fixing the discs 46, 50 to the socket wall 48a with additional fasteners, so that a combination of friction and mechanical fasteners may prevent the knee joint assembly from movement. This attachment mechanism also allows dynamic alignment of the angle of the knee joint prosthesis 10 by loosening the attachment bolt 36 that passes through the upper joint assembly or upper block 12 into the treaded inner socket disc 50. At low compression forces the discs 46, 50 are able to slip over the surfaces of the upper joint dome 12a, as well as the surfaces of the socket 48, so that the angular, rotational and translational position of the components may be adjusted. This adjustment may be made by adjusting a single point of contact at the attachment bolt 36 that is externally accessible, without the need to remove the socket 48 or knee joint prosthesis 10 from the patient.

This type of disc attachment mechanism has the advantage of distributing load over a wide area and so can be made from a relatively soft and light-weight material such as a polymer, nylon 6-6.

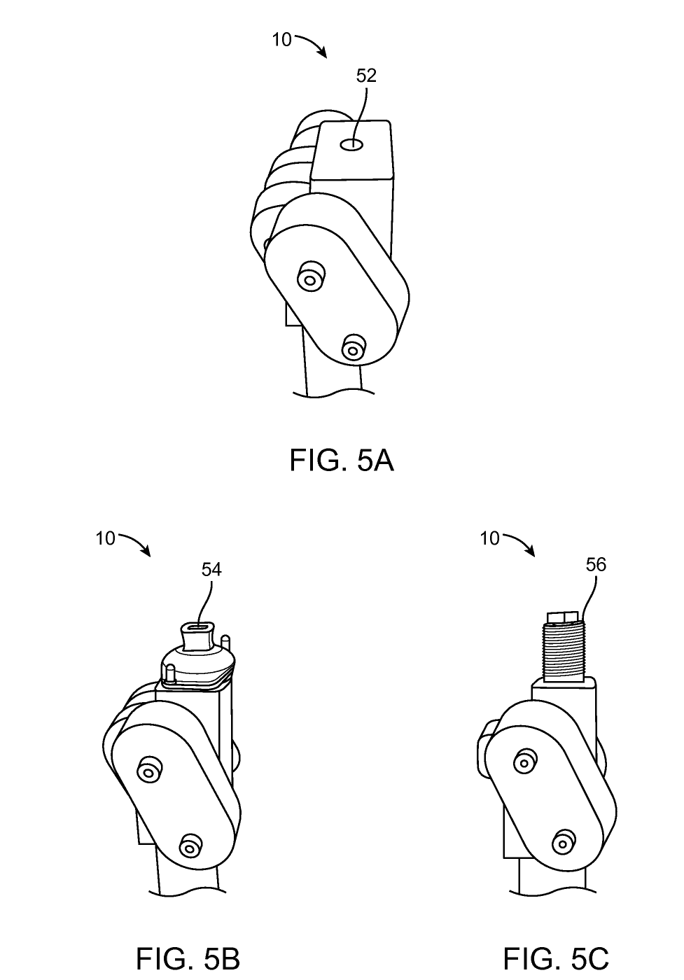

As alternatives to the previously described socket connection options, the knee joint prosthesis 10 could also use a threaded hole 52, a non-integrated adapter 54 with a large base, or a threaded rod 56, as presented in FIGS. 5A, 5B, and 5C, respectively.

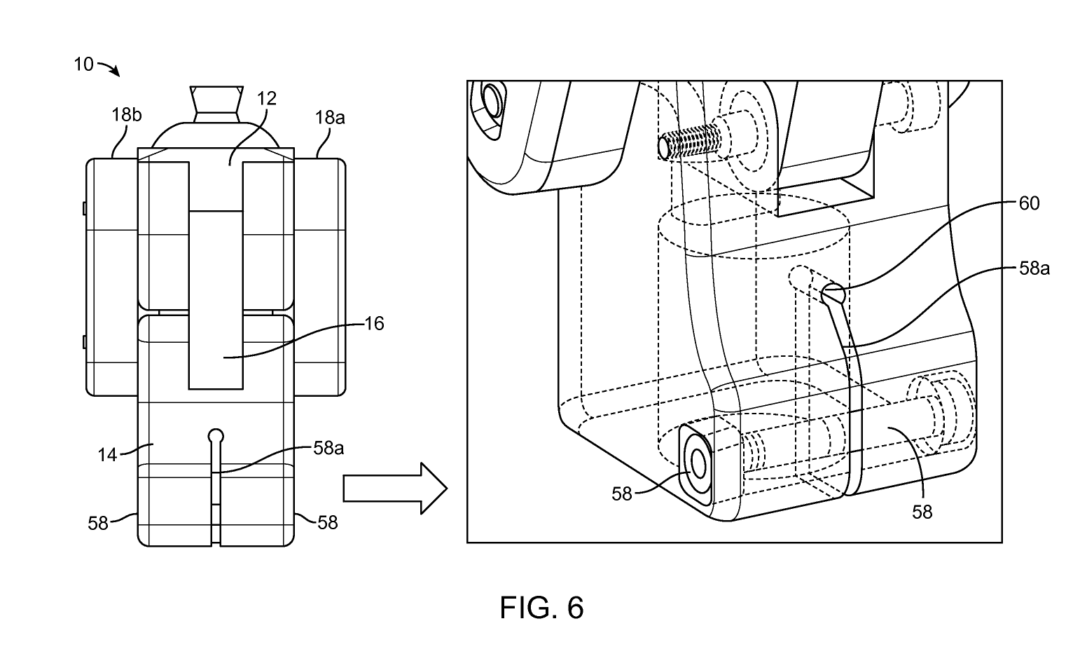

The connection mechanism for the pylon for coupling the knee joint prosthesis 10 to a foot prosthesis is shown in FIG. 6 as a split clamp mechanism 58 that is tightened around the pylon to secure it in place. A vertical slit 58a in the material of the lower block 14 allows an adjustable average diameter of a hole 60. A pylon may be inserted in the corresponding hole 60 on the lower surface of the lower block 14. Tightening a single or multiple points of adjustment may apply radial compression an inserted pylon. This type of mechanism could also include a soft intermediary surface within the clamp, such as rubber, to increase friction and absorb shock. In particular, the shock absorption of torsional loads in the prosthetic limb is a desirable feature for reducing the stresses on the patient's limb. The compliant material in between the pylon and the lower joint may thus act as a torsional stress absorber. Additionally, the split clamp 58 could make use of either a traditional bolt or a “quick-release” mechanism that allows the user to rapidly remove the knee joint prosthesis 10 from the pylon. This quick release or quick disconnect mechanism may take the form of a patient-accessible, removable pinning rod or a cammed lever on the bolt axis. Furthermore, any mechanism described above could be used at either the socket or pylon interface.

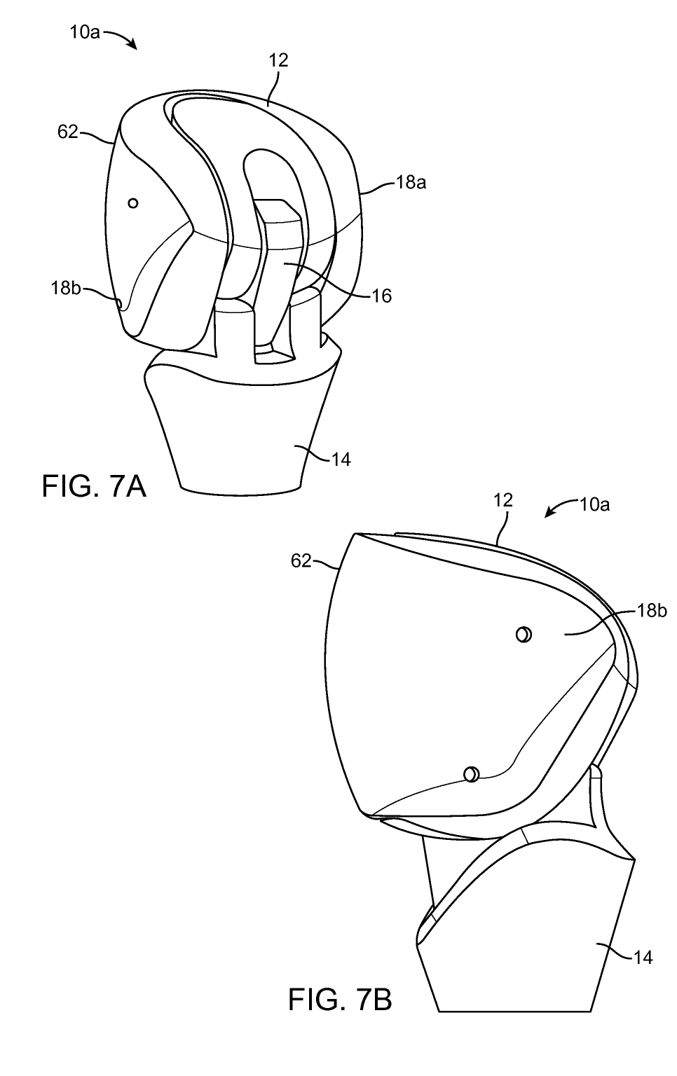

The side linkages 18a, 18b have two through-holes 22 that accept connectors 24, such as bolts, which attach the linkages 18a, 18b to the upper and lower blocks 12, 14 as best shown in FIGS. 3A and 3B. In the preferred embodiment, two side linkages 18a, 18b are shown, but one side linkage could also be used. Alternatively, the two side linkages 18a, 18b could be joined to form a single linkage 62 that wraps around the front of the knee in the same manner as a kneecap, as presented by the above knee prosthesis 10a in FIGS. 7A and 7B. The above knee prosthesis 10a is the same as the above knee prosthesis 10 in substantially every respect except that the two side linkages 18a, 18b are formed as a single piece. This single-piece design would increase the strength of the side linkages 18a and 18b, protect the internal portions of the joint prosthesis 10a while preventing the introduction of debris, and result in a more realistic cosmetic effect.

The middle linkage 16 has two-through holes that accept connectors 24, such as bolts, which attach the linkage 16 to the upper and lower blocks 12, 14. In the preferred embodiment, a single middle linkage 16 is used, but multiple linkages could be used in the same manner as the preferred side linkages.

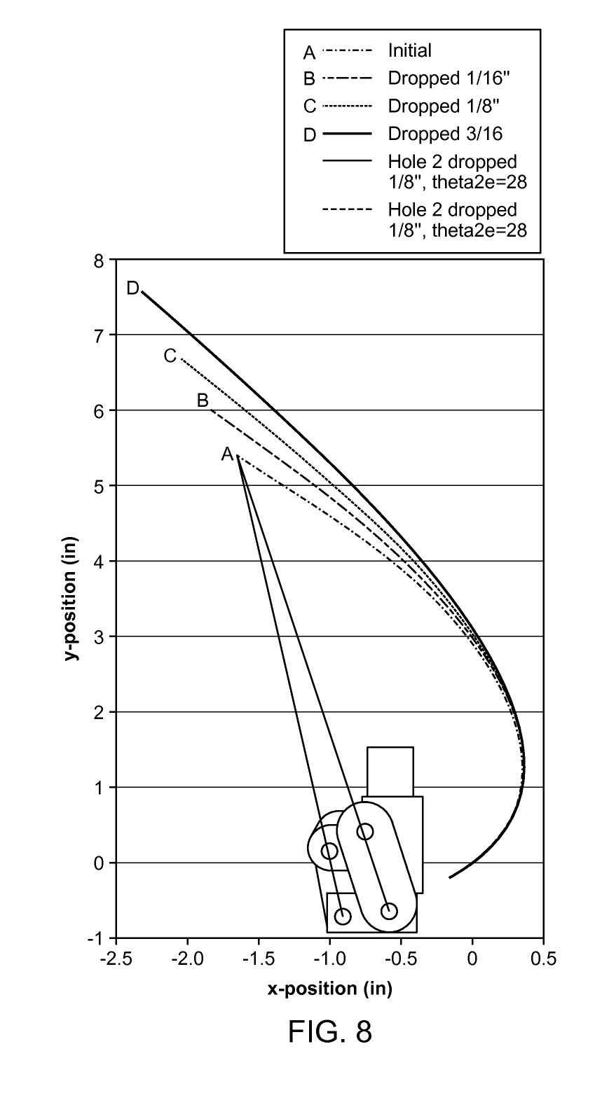

The four-bar linkage geometry used in this prosthesis 10 creates a center of rotation that is above the knee joint in stance phase, and moves downward through flexion. More specifically, the center of rotation is defined by the intersection of the lines collinear to the side and middle linkages. FIG. 8 is a graph presenting the preferred path of the dynamic center of rotation, but also includes alternate paths that could be implemented with slight changes to the hole geometry. In addition to these minor variations, the geometry could also be reflected across either the x-axis or y-axis to produce more significant modifications to the behavior of the knee joint. Use of the four-bar linkage geometry, in general, creates a knee that is very stable yet simple.

The material used for the blocks and linkages described above is preferably a strong, durable polymer such as nylon 6-6, but could alternatively be any suitable material, including any number of polymers, metals, and ceramics. Additionally, the components are all constructed of the same material, but could be constructed of different materials. Finally, the material used is preferably self-lubricating, such as an oil-filled nylon. This creates integrated bearing surfaces and eliminates the need for bushings or bearings. However, the knee joint could also be constructed of a “dry” material.

The connections between the blocks 12, 14 and linkages 16, 18a, 18b described above make use of nuts, bolts, and washers, as seen in FIG. 9. The nuts and bolts are preferably constructed of steel, and the washers are preferably constructed of nylon, but they could be constructed of any appropriate material. Alternatively, the connections between blocks and linkages could make use of protrusions that snap into recessed dimples, avoiding the need for additional hardware. Other alternatives include dowels that are part of the blocks or linkages themselves, or any other kind of mechanism that attaches the members and allows for rotation. Preferably, the knee joint prosthesis 10 avoids the use of any bearings, but they could be implemented to improve ease of rotation.

The addition of washers, or other intermediate materials between moving parts, may also be used to reduce wear and noise produced by the linkages 16, 18a, 18b rubbing against the walls of the upper block 12 and the lower block 14.

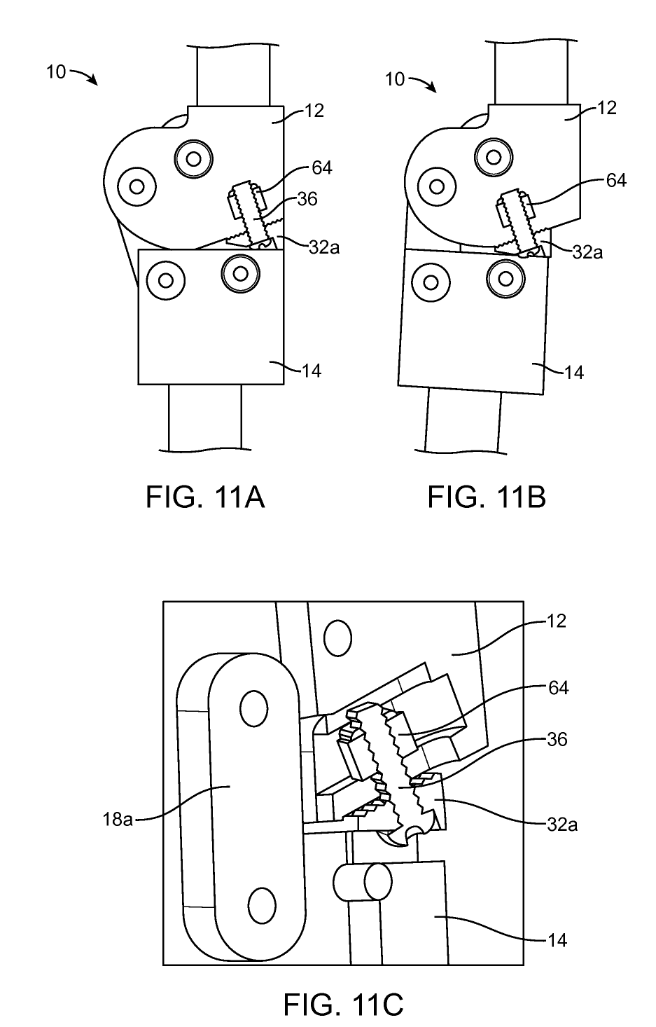

The preferred embodiment of the knee joint also has an adjustable stability mechanism, which allows the fully extended angle of the knee joint to be variable. This is accomplished by using an adjustable bolt 36, as shown in FIG. 10. The bolt 36 provides for an infinite number of positions, as determined by the depth to which it is placed. Alternatively, adjustable stability can be accomplished through the use of washers or shims that are secured between the upper block 12 and the lower block 14. For example, one or more washers or shims may be placed between the top portion 34a of the slot 34 and the bumper 32 before the bolt 36 fixedly couples the bumper 32 to the upper block 12. These shims can be either of varying sizes or in discrete thicknesses that can then be stacked to the desired height. Alternatively, the knee joint could make use of a stepped wedge 32a to adjust its inherent stability. A preferred embodiment of this wedge is presented in FIGS. 11A-11C. As seen in the illustration, the wedge 32a has steps that correspond to a finite number of adjustable positions. This stepped wedge 32a is attached to the upper block 12 with a bolt 36 that attaches to a nut 64 within the upper block 12. Alternatively, the bolt 36 could screw directly into the upper block 12 itself. Instead of interfacing with the upper block 12, the stepped wedge 32a could be attached to the lower block 14 by either of the same means.

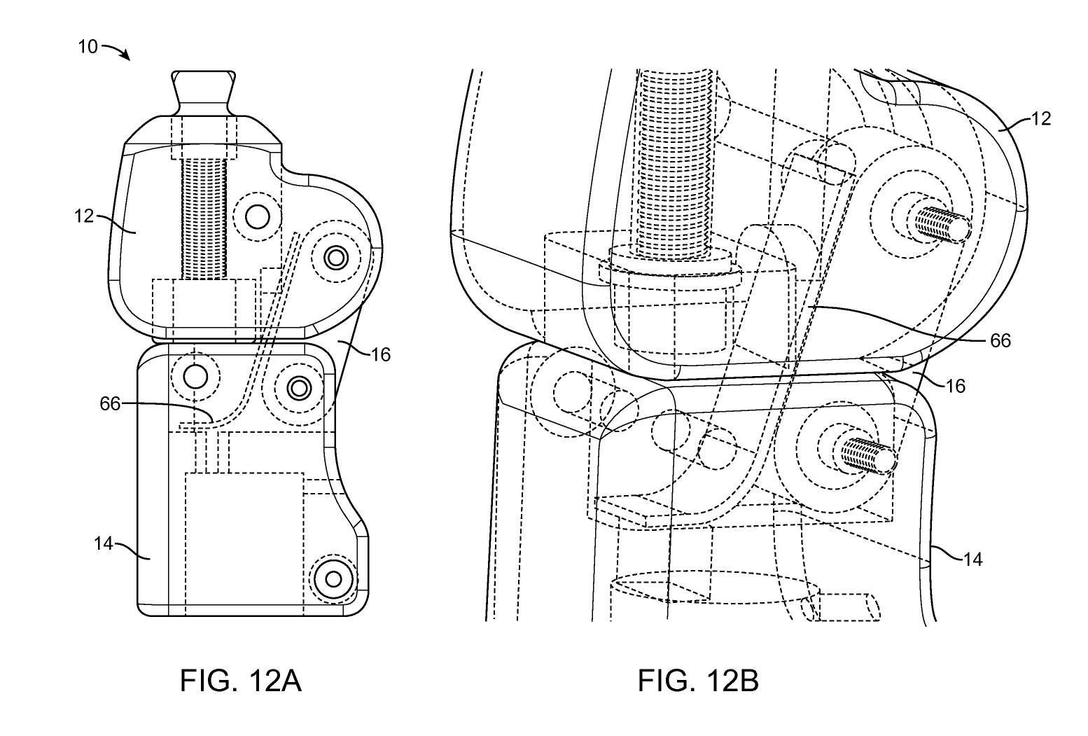

A preferred embodiment of the knee joint prosthesis 10 also includes an internal spring to assist the user during the extension phase of his gait, as illustrated in FIGS. 12A and 12B. This spring may take the form of a compliant member such as a leaf spring 66. As the knee prosthesis is flexed, this leaf spring 66 provides resistance and encourages the joint prosthesis 10 toward extension. This leaf spring 66 is preferably composed of thin sheets of steel, but could be constructed of any other suitable material. In a preferred embodiment, the leaf spring 66 is bolted to the lower block 12 and presses against the middle linkage 16. Alternatively, the leaf spring 66 could be imbedded in the lower block 12 itself to remain secure or held in place with an adhesive. In other embodiments, the leaf spring 66 could be attached to a different link, such as the upper block 12, and press against a different link, such as a side linkage 18a or 18b. As another alternative, the leaf spring 66 could be external to the knee joint. One possibility for such an embodiment could involve a leaf spring 66 on the backside of the joint prosthesis 10, attached to the upper and lower blocks 12, 14 such that the leaf spring 66 presses against the blocks 12, 14 as the knee prosthesis is flexed. Furthermore, the extension assist device could make use of a different kind of spring altogether, such as an internal compression string or an external elastomeric band that encourages the knee toward extension. Assistance of knee extension is important in order to prevent the knee joint prosthesis 10 from feeling floppy or loose, and to promote a more natural gait. Without such a mechanism, the user will generally need to jerk his leg forward to ensure that the knee joint prosthesis 10 reaches full extension and is therefore stable upon impact when stepping forward.

In another embodiment of the knee joint prosthesis 10b as shown by FIG. 14, a gravity-activated latch 68 can be used to ensure that the joint remains in extension during the stance phase of the user's gate. When in stance phase, gravity pulls the latch 68 downward against a peg 70 to prevent any flex in the joint prosthesis 10b. Once the user has raised the knee prosthesis 10b in the process of stepping forward, gravity pulls the latch 68 away from the peg 70, releasing the knee prosthesis 10b and allowing the joint prosthesis 10b to flex. In the preferred embodiment of this mechanism, the latch 68 is attached to the upper block 12 by means of a bolt 72 and rotates about a bearing 74, while the peg 70 is a simple dowel inserted into the lower block 14. However, the latch 68 could also be attached by other means, such as a pin, which would not require the use of a bearing. The peg could also be a part of the lower block 14 itself, machined into the part instead of being a separate insert. Additionally, the latch and pin locations could be located in various positions on the knee prosthesis 10, including being either internal or external to any of the blocks 12, 14 or linkages 16, 18a, 18b. Finally, although the preference is for one such mechanism, multiple latches could potentially be used. The gravity-activated latch 68 is a useful component of the above knee prosthesis 10b because it provides extra stability during the stance phase and ensures that the knee prosthesis 10b does not buckle. In another embodiment, the gravity-activation would not rely on a latch, but through weight-activated friction. In this instance, the downward force of the user locks the knee joint in place and prevents buckling.

As an alternative to a gravity-activated mechanism, a manual lock could be used. The use of a lock is advantageous when a user desires increased stability. A knee locking mechanism may involve the use of thumbscrews to tighten one of the four bolts in order to lock the joint in place. Alternatively a pin may be accessed and actuated by the patient, which may providing a mechanical locking of two or more of the rotating components of the knee joint. In its preferred embodiment, a pin is able to translate through a side linkage into a set of discrete holes in the lower block or upper block, so that knee flexion may be constrained to set angles.

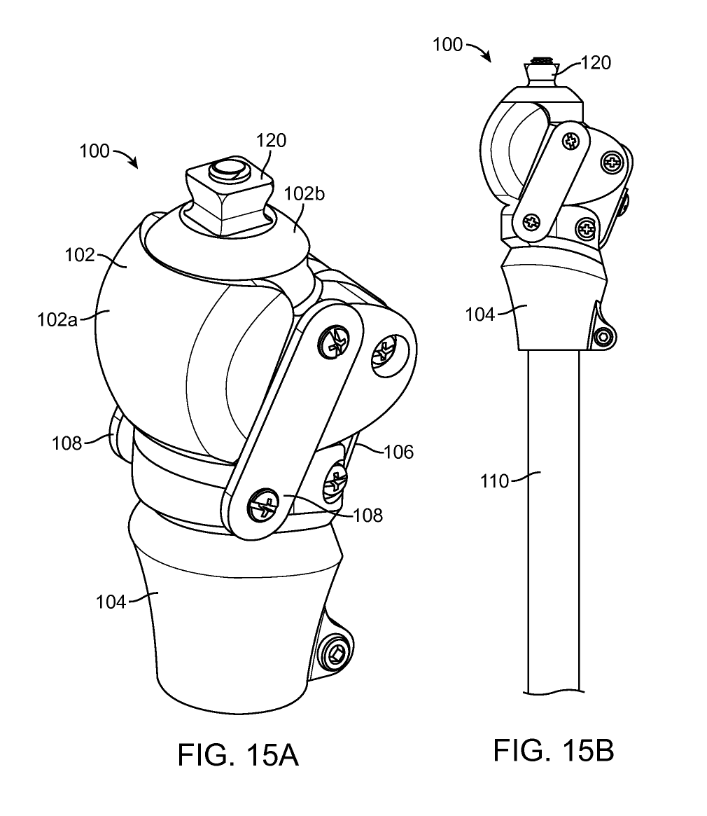

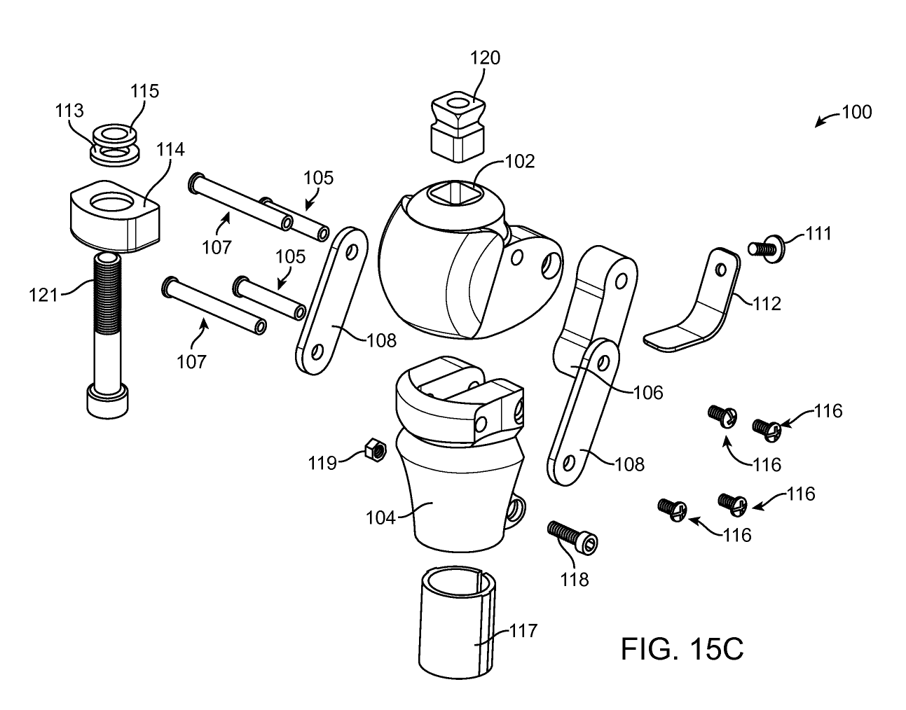

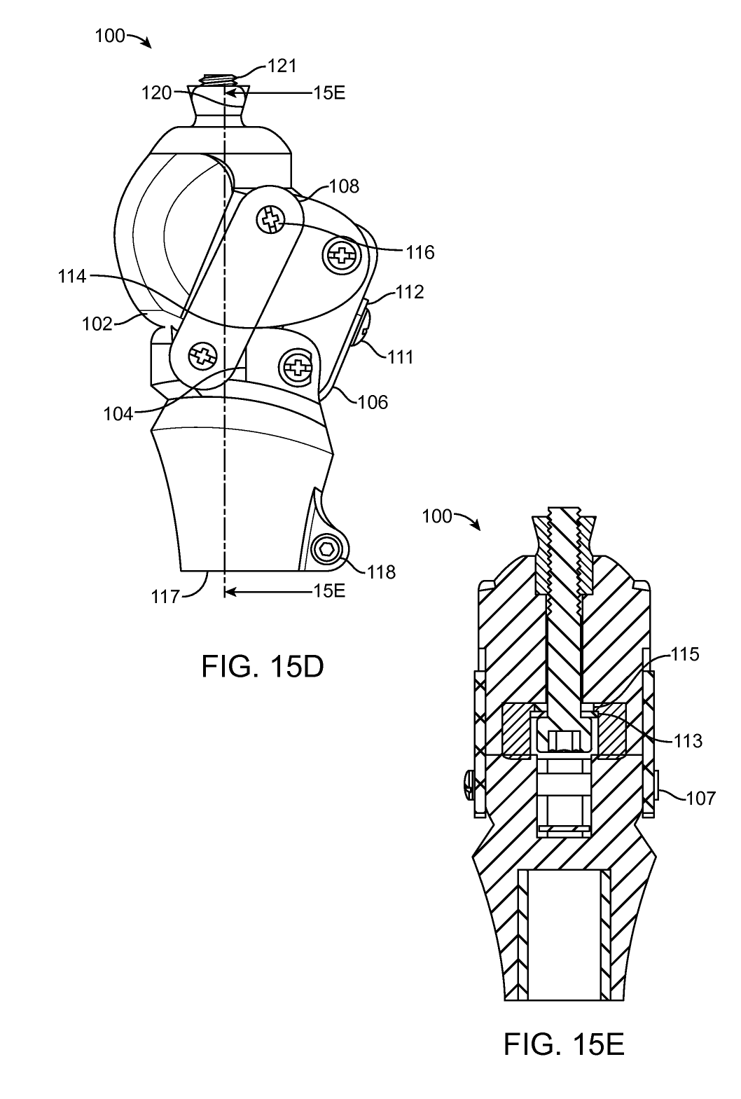

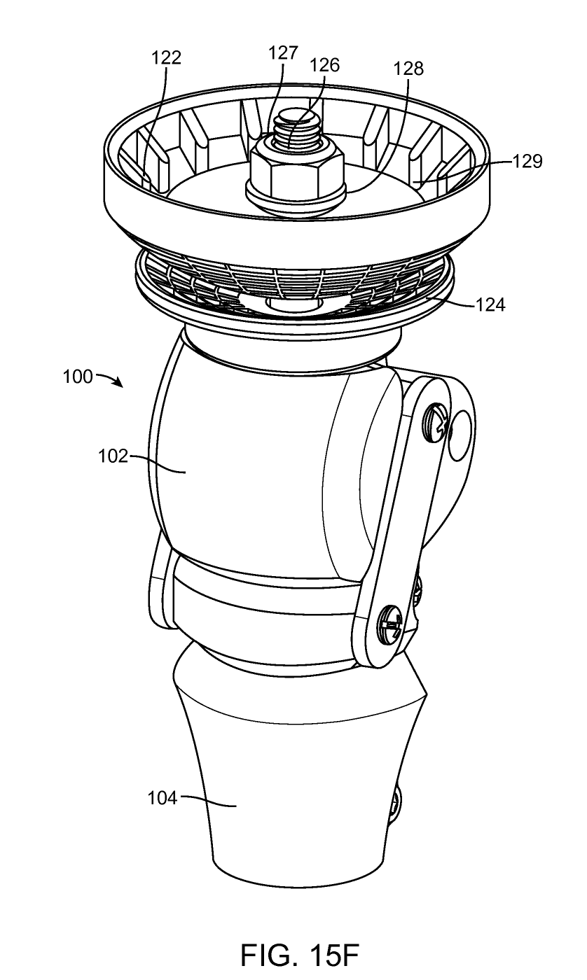

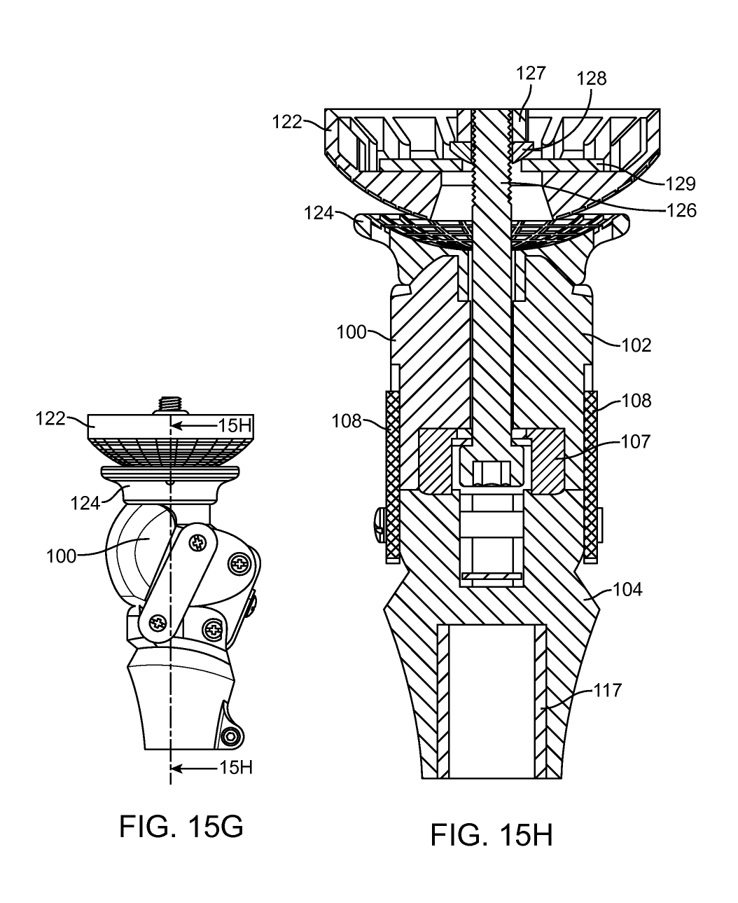

FIGS. 15A-15H show an above knee prosthesis 100 according to another embodiment of the present invention. The above knee prosthesis 100 is similar in many respects to the above knee prosthesis 10 described above, e.g., both prostheses are “polycentric.” The above knee prosthesis 100 can provided a much more rounded and aesthetic feel than the blocky above knee prosthesis 10. As shown in FIG. 15A, the knee prosthesis 100 comprises an upper block 102 having a rounded front 102a and a domed top 102b, a lower block 104 having a cylindrical main body, a middle linkage 106 pivotably coupling the middle portions of the upper block 102 and the lower block 104, two side linkages 108 pivotably coupling the sides of the upper block 102 and lower block 104, and a pyramid adapter 120 coupled to the upper block 102 and adapted to couple to a socket for the patient's leg. As shown in FIG. 15B, the lower block 104 is to be attached to a pylon 110 which attaches to a foot prosthesis.

FIG. 15C shows an exploded view of the above knee prosthesis 100. Again, many of the components of the prosthesis 100 and the prosthesis 10 described above may be similar and the prosthesis 100 primarily comprises the upper block 102, the lower block 104, the middle linkage 106, and two side linkages 108. The upper block 102 and the lower block 104 each have through-holes through which middle linkage pins 105 pass through to couple the middle linkage 106 to the upper block 102 and the lower block 104. The upper block 102 and the lower block 104 each also have additional through-holes through which side linkage pins 107 pass through to couple the two side linkages 108 to the upper block 102 and the lower block 104. Machine screws 116 secure the side linkages 108 in place relative to the upper block 102, the lower block 104, and side linkage pins 107. The prosthesis 100 further comprises a leaf spring 112 to assist the user during the extension phase of his gait. Leaf spring 112 is coupled to the middle linkage 106 with button screw 111 but may be modified in ways similar to those described above with reference to the leaf spring 66. The prosthesis 100 further comprises a bumper 114 disposed between the upper block 102 and the lower block 104 to cushion the prosthesis. The bumper 114 is coupled to the upper block 102 with a pyramid bolt 121, a bumper washer 113, and a spring lock washer 115. The bumper 114 may be similar in many respects to bumper 32 described above. The pyramid bolt 121 also couples the pyramid adapter 120 to the upper block 102. Disposed within the lower block 104 is a pylon collar adapter 117 for coupling a pylon 110 to the lower block 104 as shown in FIG. 15B. Together with bolt 118 and locknut 119, the pylon collar adapter 117 can act as a clamp to secure the pylon 110 in place relative to the lower block 104. In many embodiments, the pylon collar adapter 117 is made of polyurethane having a hardness in a range of 70-90 (preferably 85) on the shore A hardness scale and the pylon 110 is made of steel or stainless steel. For further clarity, FIG. 15D shows a side view of the prosthesis 100 with many of the above components pointed out and FIG. 15E shows a cross-section of the prosthesis 100 taken along line 15E in FIG. 15D.

Like the knee prosthesis 10 as described with reference to FIGS. 13A-13D, instead of comprising a pyramid adapter 120, the knee prosthesis 100 may be configured to attach to additional components to couple the prosthesis 100 to a socket for the patient's leg. As shown in FIGS. 15F-15H, the knee prosthesis 100 can couple to an inner socket disc 122 and an outer socket disc 124 with an elongated bolt 126, lock nut 127, a spherical washer 128, and an inner socket washer 129. FIG. 15G shows a side view of the assembly of the prosthesis 100, the inner socket disc 122, and the outer socket disc 124. FIG. 15H shows a cross-section of this assembly taken along line 15H of FIG. 15G. As shown by FIG. 15H, the elongated bolt 126 also couples the bumper 107 to the upper block 102. The outer socket disc 122 and the inner socket disc 124 can sandwich a socket for the patient's leg, e.g., socket 48 described above. In many embodiments, the middle linkage 106, the side linkages 108, the leaf spring 112, and the various pins, bolts, washers, and screws that comprise the prosthesis 100 are made of a durable and/or resilient material such as steel (spring steel for the leaf spring 112) or stainless steel while other components such as the upper block 102, the lower block 104, the inner socket disc 122, and the outer socket disc 124 are made of a relatively soft and light-weight material such as a polymer, nylon 6-6.

Forms of the above described knee joint may be used in combination with additional integrated components such as embedded sensors, and micro-processor controlled actuators and hydraulics. Sensors may include electronic components to sense the activity of the patient, number of steps, and mechanical strains and stresses within the components. Actuators and hydraulics may add additional damping and control of the knee joint to have variable stiffness at different point of the gait cycle.

As a person skilled in the art of prosthetics will recognize from the previous detailed description and from the figures and claims, modifications and changes can be made to the preferred embodiments of the invention without departing from the scope of this invention defined in the following claims.

What is claimed is:

1. An above knee prosthesis having a fully extended configuration and a fully flexed configuration, the above knee prosthesis comprising:
an upper block having a bottom face;
a lower block having a top face;
a middle linkage pivotably coupling a middle posterior region of the upper block with a middle posterior region of the lower block;
at least one side linkage pivotably coupling a side of the upper block with a side of the lower block,
wherein the center of rotation of the lower block relative to the upper block is located above the prosthesis when the prosthesis is in the fully extended configuration and moves downward as the prosthesis rotates from the fully extended configuration to the fully flexed configuration, and
wherein when the prosthesis is in use in a patient and in the fully extended configuration, a majority of the weight of the patient borne by the prosthesis is directly transferred from the bottom face of the upper block to the top face of the lower block.

2. The above knee prosthesis of claim 1, wherein the at least one side linkage comprises a first side linkage and a second side linkage, wherein the first side linkage is disposed on a first side of the upper block and on a first side of the lower block, and wherein the second side linkage is disposed on a second side of the upper block opposite the first side of the upper block and on a second side of the lower block opposite the first side of the lower block.

3. The above knee prosthesis of claim 2, further comprising a cap coupling the first side linkage with the second side linkage, the cap being disposed in front of an anterior portion of the upper block and an anterior portion of the lower block.

4. The above knee prosthesis of claim 1, wherein the middle linkage pivotably couples an internal middle posterior region of the upper block with an internal middle posterior region of the lower block.

5. The above knee prosthesis of claim 1, wherein the at least one side linkage is disposed external of a side of the upper block and a side of the lower block.

6. The above knee prosthesis of claim 1, wherein the middle linkage comprises a bar having a first end and a second end, the first end being pivotably coupled to the middle posterior region of the upper block and the second end being pivotably coupled to the middle posterior region of the lower block.

7. The above knee prosthesis of claim 1, wherein the at least one side linkage comprises a bar having a first end and a second end, the first end being pivotably coupled to the side of the upper block and the second end being pivotably coupled to the side of the lower block.

8. The above knee prosthesis of claim 1, wherein the bottom face of the upper block and the top face of the lower block comprise curved surfaces, pegs, roller bearings, or other interfaces that allow the majority of the weight of the patient borne by the prosthesis to be directly transferred from the bottom face of the upper block to the top face of the lower block.

9. The above knee prosthesis of claim 1, wherein the bottom face of the upper block and the top face of the lower block are flat.

10. The above knee prosthesis of claim 1, wherein the bottom face of the upper block and the top face of the lower block each comprise low friction surfaces.

11. The above knee prosthesis of claim 1, wherein the upper block is angled relative to the lower block at an angle of 0 degrees when the prosthesis is in the fully extended configuration.

12. The above knee prosthesis of claim 1, wherein the upper block is angled relative to the lower block at an angle of 165 degrees when the prosthesis is in the fully flexed configuration.

13. The above knee prosthesis of claim 1, further comprising a bumper disposed between the bottom face of the upper block and the top face of the lower block, the bumper being adapted to absorb shock and dampen noise when the prosthesis is placed into the fully extended configuration.

14. The above knee prosthesis of claim 13, wherein the bumper is coupled to the bottom face of the upper block.

15. The above knee prosthesis of claim 14, wherein the bumper is removeably coupled to the bottom face of the upper block.

16. The above knee prosthesis of claim 15, wherein the bumper is removeably coupled to the bottom face of the upper block by a bolt.

17. The above knee prosthesis of claim 16, further comprising a base member coupled to the top face of the upper block by the bolt.

18. The above knee prosthesis of claim 13, wherein the bumper is adjustable to adjust the distance between the bottom face of the upper block and the top face of the lower block.

19. The above knee prosthesis of claim 13, wherein the bumper is adjustable to adjust the angle of the upper block relative to the lower block when the prosthesis is placed into the fully extended configuration.

20. The above knee prosthesis of claim 13, wherein the bumper has an angled or stepped bottom face.

21. The above knee prosthesis of claim 13, wherein the bumper comprises two or more layers of materials with variable stiffness.

22. The above knee prosthesis of claim 1, wherein the prosthesis is biased to be in the fully extended configuration.

23. The above knee prosthesis of claim 1, further comprising a leaf spring coupling the upper block to the lower block and adapted to bias the prosthesis to be in the fully extended configuration.

24. The above knee prosthesis of claim 17, wherein the leaf spring is disposed internally in the upper block and the lower block.

25. The above knee prosthesis of claim 17, wherein the leaf spring comprises a flat leaf spring.

26. A system for replacing a leg of an amputee patient from above the knee, the system comprising:
the above knee prosthesis of claim 1;
a rounded socket adapted to fit to a stump of the leg of the patient;
an inner disc adapted to couple to the interior of the socket and to the above knee prosthesis to secure the prosthesis relative to the socket; and
an outer disc adapted to couple to the exterior of the socket and to the above knee prosthesis to secure the prosthesis relative to the socket, wherein a portion of socket is sandwiched between the inner and outer discs.

27. The system of claim 26, further comprising an elongated bolt adjusted to adjustably couple the upper block of the above knee prosthesis, the outer disc, the rounded socket, and the inner disc together.

28. The system of claim 27, further comprising a bumper adjustably coupled to the bottom face of the upper block of the above knee prosthesis by the elongated bolt.
